(19) United States
(12) Patent Application Publication
(10) Pub. No.: US 2014/0186820 A1
(43) Pub. Date: Jul. 3, 2014

(54) MICRO-FLUIDIC DEVICE FOR THE ANALYSIS OF A FLUID SAMPLE

(71) Applicant: Arthur Queval, Lutry (CH)

(72) Inventor: Arthur Queval, Lutry (CH)

(21) Appl. No.: 13/991,882

(22) PCT Filed: Dec. 9, 2011
(86) PCT No.: PCT/EP11/72302

§ 371 Date: Oct. 21, 2013

(30) Foreign Application Priority Data
Dec. 9, 2010 (CH) 02054/10

(51) Int. Cl. G01N 33/487

(52) U.S. Cl. CPC G01N 33/48785
USPC 435/5; 422/82.01; 422/69; 436/95; 435/34; 435/287.1; 435/4; 436/132; 436/501

(57) ABSTRACT

Use of a programmable device with capacitive touch screen for the analysis of a fluid sample as interface between a micro-fluidic device, this micro-fluidic device comprising at least one electrode and at least one micro-channel comprising an input for the introduction of the sample, and the processor of said programmable device with a capacitive touch screen.

### REFERENCE DATA

This application claims the priority of Swiss Patent Application CH-2010-2054, filed on Dec. 9, 2010, the content of which is incorporated here by reference.

### TECHNICAL FIELD

The present invention relates to a micro-fluidic device for the analysis of a fluid sample and a system for analyzing a fluid sample comprising such a device. The present invention also concerns a programmable device with a capacitive touch screen containing a processor executing a software for analyzing this sample and displaying the results of this analysis.

### STATE OF THE ART

Micro-fluidic chips are widely used in the field of diagnostics for detecting various pathologies. They enable one or several analyses to be performed quickly and accurately on a single chip using a very low volume of sample to be analyzed. These chips, also known by the name of “biochip” or “lab-on-a-chip” are generally for single use and require an interface with a computer or with other devices in order to display the results of the analysis.

In order to detect the sample, different methods are used, among which optical, magnetic, electromagnetic, piezoelectric, resistive and capacitive, amperometric and coulometric detection methods.

The detection signal is then converted into an electric signal to undergo processing (filtering, sampling etc.) and enabling the characteristics of the fluid sample to be analyzed, for example its composition, temperature, fluid speed, viscosity, conductivity, pH etc.

Whatever the detection method used, each micro-fluidic chip and its interface are generally developed for a specific application, which limits the chip's modularity and flexibility.

The integration of the analysis electronics into a known micro-fluidic chip increases its price. This chip is furthermore for single use since it is difficult to sterilize it, due to the small dimension of its micro-channels for sampling the fluid.

Techniques have been developed to increase the modularity of micro-fluidic devices using existing technologies. Document US20060166357 describes a micro-fluidic support whose elements, such as valves, pumps etc., which need to exert a pressure onto the micro-channels are activated by an external element such as an interactive touch screen through which it is possible to exert pressure onto the channels. In particular, the support functions with a device for Braille writing which can be controlled by a computer through a simple text editor. However, the touch device only serves for entering commands.

WO2008117223 describes a biochip integrating actuators and/or sensors placed in a matrix fashion so as to interact with the micro-fluidic device. Each actuator/sensor can be activated individually thanks to an active matrix of transistors made using thin-film technology. However, the interpretation and analysis of the results are performed on an external device. Furthermore, the electronics must be directly integrated onto the chip, which thus increases the biochip's manufacturing costs.

### BRIEF SUMMARY OF THE INVENTION

One aim of the present invention is to propose a device for the analysis of a fluid sample that is free from the limitations of the known micro-fluidic chips.

Another aim of the invention is to propose a device for the analysis of a fluid sample that is more modular, flexible and less costly than the known solutions.

According to the invention, these aims are achieved notably by means of a micro-fluidic device according to claim 1, by means of a programmable device with capacitive touch screen according to claim 11, by means of a method for the analysis of a fluid sample according to claim 18, by means of a computer-readable non-transient data-storage means according to claim 20 and by means of a system according to claim 21.

This present invention uses the capacitive detection methods of a programmable device with a capacitive touch screen as interface between the fluid sample and an analysis tool. The idea proposed here is to use a programmable device with a capacitive touch screen, for example a touch tablet, as interface and also as computing unit for the analysis of a micro-fluidic device, for example of a micro-fluidic chip.

The capacitive touch screen of the programmable device can be assimilated to a peripheral operating as input/output that could be used as detection interface. In fact, programmable devices with capacitive touch screens are nowadays produced in industrial quantities and are affordable for a mass consumption market. Furthermore, these devices enjoy increased computing power with powerful micro-processors and, thanks to the progress of the micro-electronic industry, have a low energy consumption.

In one variant embodiment, the programmable device with capacitive touch screen can be configured to enable the analysis of a micro-fluidic device of different types or of several micro-fluidic devices, of the same type or of different types. In another variant embodiment, several programmable devices with capacitive touch screen can be used in parallel and communicate the results of the analyses with one another.

Unlike known solutions, which require a sensor, a communication bus with an analysis and/or display unit and such an analysis and/or display unit, the present invention combines the three functions (detection, analysis and display) into a single one thanks to the use of a programmable device with capacitive touch screen.

Furthermore, the use of a programmable device with capacitive touch screen that already exists on the market makes it possible not only to reduce the development costs but also to reach a wider market.

### BRIEF DESCRIPTION OF THE FIGURES

Examples of embodiments of the invention are indicated in the description illustrated by the attached figures in which:

FIG. 1 illustrates a section view of the programmable device with a capacitive touch screen interfaced with the micro-fluidic device.

FIG. 2 illustrates an equivalent electric diagram of the programmable device with capacitive touch screen and of the micro-fluidic device.

FIG. 3 illustrates a three-dimensional view of an embodiment of the programmable device with capacitive touch screen.

FIG. 4 illustrates an embodiment of the micro-fluidic device according to the invention.

FIG. 5 illustrates a section view of another embodiment of the programmable device with a modified capacitive touch screen interfaced with the micro-fluidic device.

### EXAMPLE(S) OF EMBODIMENTS OF THE INVENTION

The present invention concerns a micro-fluidic device 1 for the analysis of a fluid sample using as an interface, as a means for analyzing the sample and as a means for displaying the results of the analysis a programmable device with a capacitive touch screen 2, i.e. based on a so-called capacitive technology. The fluid of the sample can be a liquid, for example but in a non-limiting way, blood, urine, serum, blood plasma, saliva, secretions or a gas, for example but in a non-limiting way, breath.

Programmable devices with a current touch screen are based mainly on two technologies: the one being resistive, the other capacitive.

Resistive technology consists in superimposing two transparent conducting films separated by a slight gap. During an action on the device, the pressure exerted by the touch creates a mechanical deformation of the upper film and makes it enter into contact with the lower film, thus creating an electric contact between these two films. The measurement of the resistance enables the contact to be detected and localized. The advantage of this technology is the possibility of using both objects as well as fingers as long as the pressure is sufficient to sufficiently deform the upper film. By contrast, these types of devices will have the tendency of being worn and scratched more easily due to the low mechanical resistance of the upper film.

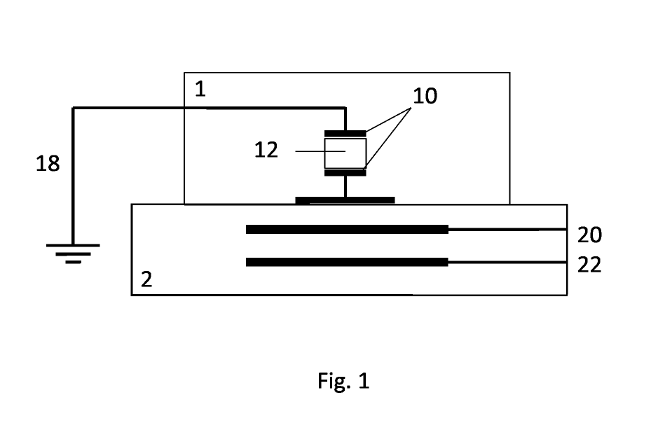

Capacitive technology consists in countersinking two arrays of transparent electrodes composed of ITO (Indium Tin Oxide), the first array being composed of detection electrodes 20 and the second by excitation electrodes 22, visible in cross section in FIG. 1, with the two arrays being placed perpendicularly one relative to the other in the touch device. During touch on the screen of the device with a finger or any conducting object, the electric field generated by the excitation electrodes 22 is disturbed and part of the charges supplied by the array of excitation electrodes 22 go through the finger and into the earth. The charge deficit is then detected by the array of detection electrodes 20 which identifies the location of the touch by means of a processor.

The advantage of this technique relative to the resistive one is that there is no mechanical deformation of the device's screen, which is thus longer-lasting and robust thanks to the use of resistant glass. Another considerable advantage is the possibility of detecting several touches simultaneously (“multi-touch”), which is currently not possible with a resistive-type technology.

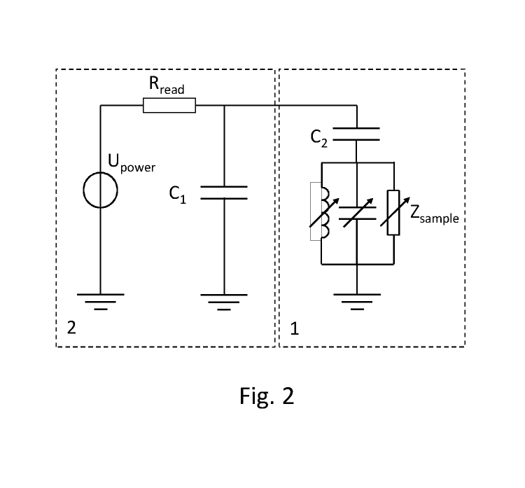

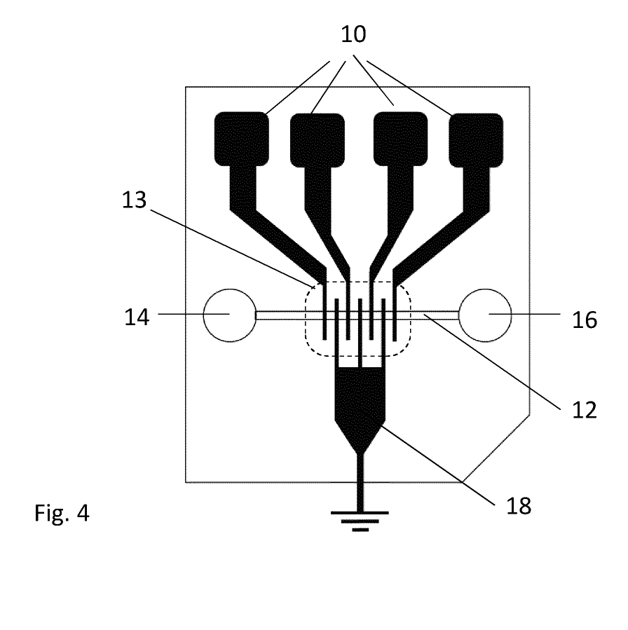

According to the invention, a device with a touch screen 2 using capacitive technology can capitalize on the variation in impedance Zₛₐₘₚₗₑ, visible in FIG. 2, caused by a micro-fluidic device 1. In this case, the variation in impedance, for example a variation in capacity, will not be generated by the touch of a finger but by the variation of the composition or of the characteristics of a sample placed on a surface 121 between two electrodes 10 of the micro-fluidic device 1 (FIGS. 1 and 4).

Advantageously, the programmable device with capacitive touch screen 2 comprises a processor executing a software for achieving a detection of the micro-fluidic device 1. The detection, thanks to a software stored in the programmable device 2, makes it possible to interpret the quantity of charges missing and thus to analyze the fluid sample. The quantity of charges lost will be analyzed depending on the position on the capacitive touch device 2 and as a function of the time. Furthermore, if the programmable device with capacitive touch screen 2 is also “multi-touch”, it enables several reading zones to be managed simultaneously for multiple analyses.

According to the invention, a micro-fluidic device 1 is arranged so as to interface directly on the display surface 24 of a programmable device with capacitive touch screen 2, for example a touch tablet or a multifunction telephone (“smartphone”) which will detect the presence of the micro-fluidic device 1, will analyze the information coming from the micro-fluidic device 1 according to the composition of the sample to be analyzed and will display the results of this analysis on the capacitive touch screen.

The programmable device with capacitive touch screen 2 advantageously comprises a memory executing a software for analyzing the charge variations caused by the micro-fluidic device 1 on the capacitive touch screen of the programmable device 2 and for deducing the analysis results of the fluid sample.

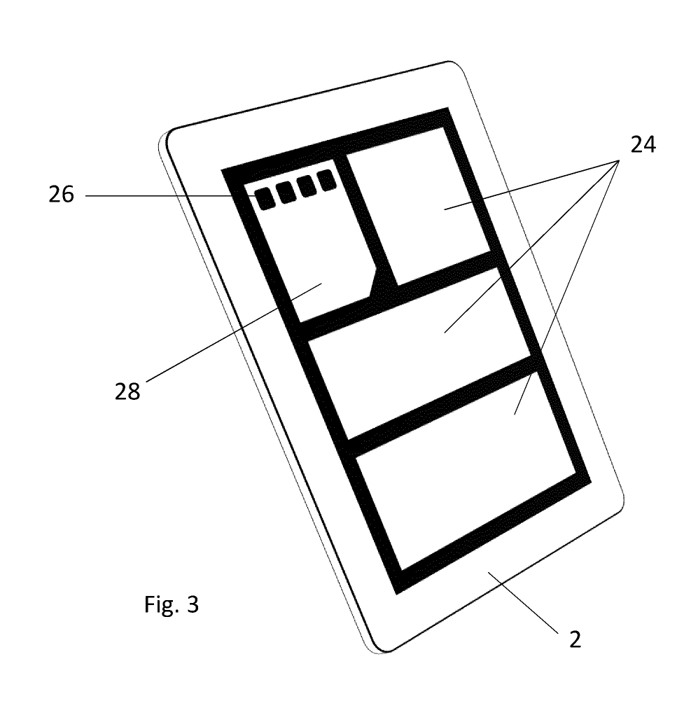

According to the invention, the sample would first be injected into the micro-fluidic device 1, which is single use, then simply placed on a reading zone or surface 28 defined on the display surface 4 of the programmable device with capacitive touch screen 2, visible in FIG. 3. This surface 28 of the programmable device designed to interface with the micro-fluidic device comprises a surface 26 designed to interface with at least one electrode 10 of the microfluidic device 1. The surfaces 26 and 28 will be displayed on the programmable device 2 before the micro-fluidic device 1 is placed. The programmable device with capacitive touch screen can comprise several surfaces 28.

After having analyzed the information coming from the micro-fluidic device 1 according to the composition of the sample to be analyzed, the programmable device with capacitive touch screen 2 displays the results of the analysis.

The micro-fluidic device 1 is composed of one or several electrodes 10 to interface with the programmable device with capacitive touch screen 2 and one or several inputs/outputs 14 respectively 16 of at least one micro-channel 12 for the introduction of the sample to be diagnosed (FIG. 4). The detection surface 13 is situated in the part of the micro-channel 10 that is superimposed over the electrode array 10 in a comb-like fashion. The analyte to be tested is placed on one or several surfaces 121 of the channels 12 opposite the detection zone 13.

The micro-fluidic device 1 is disposable and single use. In one embodiment, it is made of polymer thanks to micro-manufacturing methods, for example thermoforming methods, which are mastered and well adapted for a low cost industrial production.

In another variant embodiment, the micro-fluidic device 1 can be associated with an external electronics for signal amplification or for actuating one or several of its parts, for example micro-valves, pumps.

In a variant embodiment, the micro-fluidic device 1 is made of several parts, of which one can be single use whilst the other can be either also single use or preferably reusable. In one embodiment, the micro-fluidic device 1 comprises a disposable portion with one or several surfaces 121 for the sample to be tested as well as electrodes and an intermediary reusable device designed for being placed onto the touch screen. The disposable portions is plugged in and/or connected electrically to the intermediary device, so that the electrodes of the disposable portion are electrically connected with the electrodes of the intermediary device. The intermediary device is thus not in contact with the fluid to be tested and can be reused. The electrodes of the intermediary device are then in electric contact with the electric portion of the disposable portion, whose impedance depends on the fluid to be tested. The capacitive electrodes of the touch screen 2 detect this variable impedance. The micro-fluidic device 1 ensures an electric contact of the electrodes 10 with the mass 18, enabling the electric charge circulation circuit to be closed again.

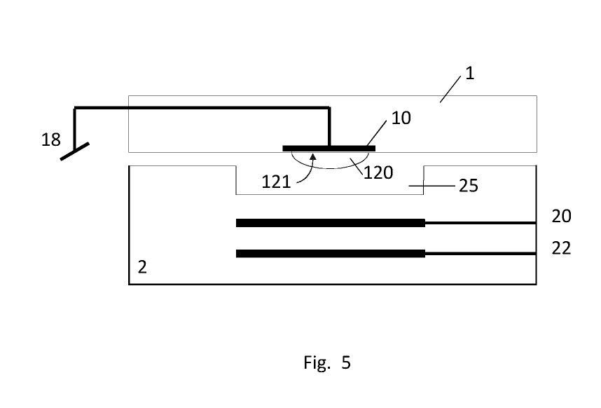

In one variant embodiment, the fluid is in direct contact with the capacitive screen. This can be achieved in several ways:
In the embodiment illustrated in FIG. 5, the channel, or at least one or several slots 25, is made directly in the screen in order to reduce the electrode-to-fluid distance and thus increase the signal and/or reduce the noise. The micro-fluidic device closes off the channel or slot to interface with the screen. The analyte can be in contact with the micro-fluidic device; or with the touch screen; or with the two surfaces simultaneously.
In another variant embodiment, not represented, an open channel is provided under the lower surface of the micro-fluidic device and which is closed off when pressed flush with the non-modified surface of the screen.
In a third embodiment, the channel is made partly in the screen and partly in the micro-fluidic device.

In the variant of FIG. 5, the micro-fluidic device 1 works with a programmable device with modified capacitive touch screen 2. In this example, the surface of the touch device comprises one or several slots 25, for example slots of rectangular or round shape or in the shape of channels in rows or arrays for example. These slots are directly engraved or produced in the outer surface 24 of the glass covering the touch screen, with a depth for example of less than 2 millimeters. The slots 25 make it possible to place the analyte (drop or bead of liquid or gas) to be tested placed directly onto a surface 121 under the lower surface of the micro-fluidic device 1, which in this example is plane. In another embodiment, the liquid to be analyzed is placed directly onto the modified capacitive screen 2, for example directly into the slots 25, or at the extremity of a channel that conducts the fluid into these slots by capillarity. The liquid can also be introduced at the extremity of a channel into the micro-fluidic device and flow by capillarity or gravity into the slot 25 engraved in the screen of the device 25.

In a variant embodiment, not illustrated, the slots are constituted by holes or blind holes in a plate that is pressed flush onto the touch surface of the touch device 2 in order to create an overthickness enabling sufficiently deep slots to be created so as to accommodate drops of liquid to be tested above the touch device.

It is also possible to create on this overthickness plate, or to display on the touch screen 2, markings for correctly positioning the micro-fluidic device 1.

This device is thus easier to use for certain applications; it is sufficient to place a drop of liquid at one or several predefined places under the lower surface of the microfluidic device 1 and then to apply this micro-fluidic device at the correct place against the screen of the touch device 2 for the drops of liquid to be tested to be placed in the slot or slots 25 provided to this effect during the test. It is also possible to provide channels through the micro-fluidic device 1 in order to bring by capillarity the liquid (or the gas by inflow) introduced in another place into the predefined surfaces opposite the slots 25.

The micro-fluidic device of FIG. 5 can also, in a non-represented embodiment, comprise a lower surface that is not plane, for example with slots opposite the slots 25, so as to be able to receive greater volumes of analytes without having to engrave slots that are too big into the device 2.

FIG. 2 illustrates an equivalent electric diagram of the interface between the programmable device with capacitive touch screen 2 and the micro-fluidic device 1: the programmable device 2 is schematized with a voltage generator U_{power}, a capacitance C₁ and a resistance R_{read}. The coupling between the programmable device with capacitive touch screen 2 and the micro-fluidic device 1 is schematized with a capacitance C₂. The micro-fluidic device 1 is schematized with an impedance Zₛₐₘₚₗₑ, which can vary according to the sample to be analyzed and which is connected to the earth.

A software stored in a memory of the programmable device with capacitive touch screen 2 makes it possible to directly read and analyze the data read on the micro-fluidic device 1 and then to display the results on the capacitive touch screen of the device 2. The software is thus capable of quantifying the variations in impedance of the programmable device 2 created by the micro-fluidic device 1 and of determining from it one or several characteristics of the sample to be analyzed.

In a variant embodiment, the software can analyze several micro-fluidic devices 1 simultaneously as long as the surface of the capacitive touch screen of the programmable device 2 and the computing power of its processor allow it.

In a variant embodiment, the software makes it possible to control the touch screen control, in order to avoid or modify the filtering usually performed by this controller to remove the variations in impedance that are not produced by a finger of the user. In a variant embodiment, a new firmware is loaded into this controller. In a variant embodiment, the software makes it possible to avoid or modify the software processing of the touches that is usually performed, in addition to the processing by the touch screen controller, by the operating system of the device 2.

The controller driving the capacitive touch screen could operate in hybrid mode: a “standard” mode and a “sample(s) analysis” mode. In the “standard” operating mode, the controller functions as in a conventional touch device, for example a touch tablet or a smartphone. It is capable of distinguishing intentional touches (one or several fingers) from involuntary touches (palms, ears, cheeks etc.). In the “sample(s) analysis” mode, the controller is capable of detecting variations in charges of the screen interfaced with the micro-fluidic device in one or several specific detection zones of the screen.

In one embodiment, an additional controller designed for the “sample analysis” mode only can be implemented in to a conventional controller operating in “standard” mode for processing the signals. The switch from one mode to another can occur either automatically or manually through an interface software executed by the device 2.

The reading surfaces 26 of the electrodes and of the micro-fluidic device 1 as well as their number can be configured according to the micro-fluidic device 1 that is used.

The software also provides the possibility of exporting the result data directly through the programmable device with capacitive touch screen 2. For this purpose, the programmable device 2 comprises means wired and/or wireless means for exporting the results of the analysis to an external computer, for example using a 3G/4G, Wifi, Bluetooth, USB or other interface.

The data exported by Internet or other communication modes can be reinterpreted and stored by competent personnel. The data can be indexed on a user account to retrace the history of the analyses.

In one variant embodiment, the programmable device with capacitive touch screen 2 also comprises means for storing the results of the analysis.

In another variant, the results of the analysis will not be displayed on the capacitive touch screen of the programmable device 2 but will be sent to an external device on which they will be displayed.

Since the micro-fluidic device 1 comprises no or only minimal detection electronics, the production cost is reduced compared with other integrated systems. The disposable part can be decoupled from the possible external electronic part so as to reduce the former's manufacturing costs.

Since the analysis is performed on an existing programmable device with capacitive touch screen, for example a touch tablet or smartphones, no dedicated analysis system consequently needs to be developed. Furthermore, using a programmable device with capacitive touch screen 2 to interface with a microfluidic device 1 is a simple diagnostic method that can attract wider markets than more robust but also more expensive devices.

The acquisition, analysis and result data display software can be commercialized through online sales platforms. Several software packages can be marketed depending on the needs of the different users (private persons, medical personnel, medical doctors, humanitarian organizations).

The micro-fluidic device 1 in one variant embodiment comprises a combination of numbers and/or shapes of electrodes enabling the programmable device with capacitive touch screen 1 to identify the type or types of analysis to be performed and/or to identify the user of the micro-fluidic device 1. In a preferred embodiment, the electrodes have the shape of a one-dimensional or two-dimensional barcode. In another variant, the micro-fluidic device 1 comprises an RFID tag that can be read by an RFID reader integrated in the programmable device 2.

In another variant embodiment, the micro-fluidic device 1 comprises a coating to enhance the interaction with and analysis of the fluid sample. For example, enzymes or molecules can be previously provided on the electrode or electrodes 10 and/or the micro-channel or micro-channels 12. More generally, the surface 121 for placing the analyte, for example the electrode on which it is placed, or another portion of the micro-channels 12, can undergo a chemical functionalization operation. The aim of the chemical functionalization of the surface is to recognize only specific molecules (contained in the sample to be analyzed) that will adhere to the surface. The variation in capacitance is then due to this specific molecule (and not others present in the sample) being recognized and adhering.

Different surfaces 121 in a same micro-fluidic device 1 can have different types of functionalization to react each specifically to a precise type of molecule for different analyses.

In another variant embodiment, the results of the analysis on the fluid sample can be combined with data from the programmable device 2, which can include for example a GPS module, for performing statistical analyses and/or follow the geographic evolution of these results.

Non-exhaustive and non-limiting examples of applications of the present invention are
measurement of the level of glucose in the blood for diabetics;
measurement of water pollution or food contamination (virus, bacteria);
alcohol test for measuring the level of alcohol in the blood or in the breath;
detection of explosive substances;
identification of allergies from a blood sample.

### REFERENCE NUMBERS USED IN THE FIGURES

1 micro-fluidic device
10 electrode
12 micro-channel
120 volume of analyte to be tested
121 surface for placing the sample to be tested
13 detection surface
14 input of the micro-channel
16 output of the micro-channel
18 electric contact with mass
2 programmable device with capacitive touch screen
20 detection electrode
22 excitation electrode
24 display surface of the capacitive touch device
25 slot or channel in the surface 24
26 surface of the capacitive touch device designed for interfacing with at least one electrode of the micro-fluidic device
28 surface of the capacitive touch device designed for interfacing with the micro-fluidic device
U_{power} power voltage of the capacitive tactile device
C₁ capacitance of the capacitive tactile device
R_{read} resistance of the capacitive tactile device
C₂ coupling capacitance between the capacitive tactile device and the micro-fluidic device
Zₛₐₘₚₗₑ impedance of the fluid sample

1. Micro-fluidic device for analyzing a fluid sample, comprising:
at least one electrode;
at least one surface close to said electrode, for placing said fluid sample;
wherein said at least one electrode is arranged so as to produce a measurable capacitance variation on the touch screen of a programmable device with capacitive touch screen when said device is placed on said touch screen, so as to enable the analysis of said fluid sample.

2. The micro-fluidic device of claim 1, wherein said surface is formed on at least one micro-channel comprising an input for the introduction of said sample.

3. The device according to claim 1, made of polymer by means of a micro-manufacturing method.

4. The device according to claim 1, comprising at least one micro-valve and/or at least one pump.

5. The device according to claim 4, designed for being coupled with an external electronics for signal amplification and/or for actuating said at least one pump.

6. The device according to claim 2, comprising a detection surface between said input and an output of said micro-channel.

7. The device according to claim 2, comprising at least one said electrode on the side of said micro-channel opposite said programmable device and at least one said electrode on the opposite side of said micro-channel.

8. The device according to claim 1, comprising at least one outer surface opposite said electrode on which said sample is intended to be placed directly for the test.

9. The device according to claim 1, wherein said surface enabling said fluid sample to be placed is functionalized chemically so as to enable the adhesion of specific molecules contained in the sample to be analyzed.

10. The device according to claim 1, comprising a portion for single use comprising said surface for placing said fluid sample to be placed, and an intermediary device in electric contact with said single use portion and designed to be placed onto said touch screen.

11. A programmable device with capacitive touch screen comprising:
at least one display surface for displaying the results of the analysis;
at least one portion of said display surface, designed for interfacing with the device of claim 1;
at least one processor; and
a memory storing a software that can be executed by said processor in order to analyze the variations in charge caused by said device on said surface and for deducing said analysis results from said sample.

12. The device according to claim 11, wherein said software comprises portions of code for configuring the position and dimensions of said at least one surface.

13. The device according to claim 11, wherein said software comprises portions of code for displaying said at least one display surface and at least one surface designed to interface with at least one electrode of the micro-fluidic device before the micro-fluidic device is placed on said programmable device.

14. The device according to claim 11, wherein said software comprises portions of code for exporting said results to an external computer.

15. The device according to claim 11, wherein said software comprises portions of code for storing said results.

16. The device according to claim 11, constituted by a touch tablet or a touch smartphone comprising a program for fluid analysis on the basis of variations in said capacitance.

17. The device of claim 16, comprising slots engraved into said display surface for receiving said samples.

18. A method for analyzing a fluid sample comprising the following steps:
placing said sample on a surface of a micro-fluidic device;
placing said micro-fluidic device on the touch screen of a programmable device with capacitive touch screen;
analyzing the variations in charge caused by said micro-fluidic device on said capacitive touch screen and for deciding analysis results of said sample; and
displaying on said capacitive touch screen the results of the analysis.

19. The method of claim 18, wherein said sample is placed on said surface by being introduced in an input of at least one micro-channel of the micro-fluidic device.

20. A computer-readable non-transient data-storage means, comprising instructions executed by a computerized control unit for the analysis of a fluid sample introduced in an input of at least one micro-channel of a micro-fluidic device interfaced with a programmable device with capacitive touch screen, wherein said instructions make said control unit execute a method comprising the following steps:
analyzing the variations in charge caused by said micro-fluidic device on said capacitive touch screen and deducing the analysis results of said sample; and
displaying on said capacitive touch screen the results of the analysis.

21. A system comprising a micro-fluidic device designed for analyzing fluid samples and a programmable device with capacitive touch screen designed for displaying the results of said analysis, wherein the micro-fluidic device is designed so as to modify the capacitive charges on said touch screen depending on said fluid sample.
